(19) United States
(12) Patent Application Publication
(10) Pub. No.: US 2002/0044912 A1
(43) Pub. Date: Apr. 18, 2002

(54) Composition for remineralization of a tooth

(75) Inventors: Yoko Ishihara, Itabashi-ku (JP); Eiichi Yoshii, Itabashi-ku (JP); Kazuo Hirota, Itabashi-ku (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: GC Corporation, Itabashi-ku (JP)

(21) Appl. No.: 09/898,055

(22) Filed: Jul. 5, 2001

(30) Foreign Application Priority Data
Aug. 8, 2000 (JP) 2000-239529

(51) Int. Cl. A61K 7/16

(52) U.S. Cl. 424/57

(57) ABSTRACT

A composition for remineralization of a tooth, adapting to a tooth surface demineralized due to dental caries and forming a compound within the tooth to gradually release a phosphate ion and a calcium ion therein, is comprised of a solution A to be first applied to a tooth surface, containing 1 to 30% by weight of a calcium salt, with the remainder being a volatile solvent; and a solution B that is an aqueous solution containing 1 to 30% by weight of a phosphate and preferably a fluoride in an amount of 0.0001 to 5% by weight, where at least one of the solution A and the solution B preferably further contains a surfactant in an amount of 0.0005 to 1 % by weight.

### BAKCGROUND OF THE INVENTION

1. Field of the Invention

The present invention relates to a composition for remineralization of a tooth, which is aimed to accelerate remineralization of a tooth by applying it to a tooth surface demineralized due to dental caries and forming a compound within the tooth, from which a phosphate ion and a calcium ion are then gradually released.

2. Description of the Conventional Art

In an oral cavity, a tooth repeats demineralization and remineralization. A dental caries refers to a disease resulted from the matter that a balance between demineralization and remineralization is lost, whereby the balance is greatly inclined to the demineralization side. Thus, if an intraoral circumstance were regulated such that the balance is inclined to the remineralization side, it would become possible that the tooth is again mineralized to stop the progress of the dental caries and apply a remedy.

As factors for introducing this remineralization, are enumerated the amount of saliva and the presence of a fluoride ion. But, first of all, it is important that sufficient amounts of a phosphate ion and a calcium ion as constitutional substances of a tooth is present in a demineralized site.

As a method for supplying the phosphate ion and calcium ion, for example, Japanese Patent Laid-Open No. 94187/1974 discloses “Processes and compositions for remineralization of dental enamel”; and International Patent Publication No. 2000-504037 discloses “Product and method for remineralization and prevention of the demineralization of the teeth”.

In the former, it is disclosed that a tooth surface is treated successively with solutions containing individually a calcium ion and a phosphate ion, whereby a precipitate formed by the reaction thereof is provided as a supply source for phosphoric acid and calcium. In the latter, it is disclosed that in order to prevent a product from immediate precipitation, a tooth surface is treated with an aqueous solution containing both of a phosphate and a fluoride and a solution containing a water-soluble calcium salt, and after these have been diffused under a surface layer, a compound is formed.

Further, for example, Japanese Patent Laid-Open No. 217904/1992 titled with “Odon to therapeutical material” and Japanese Patent Laid-Open No. 255029/1993 titled with “Composition for treating tooth surface” disclose a method for forming a sparingly soluble precipitate or hydroxyapatite on a tooth surface.

However, in the case where the composition as disclosed in each of the above-cited patent publications is used, the following problems arose. That is, during the formation of a calcium phosphate compound or fluoroapatite, the second solution cannot well penetrate into an enamel or dentinal tubule so that the reaction occurs only in the surface layer of the tooth. As a result, the formed compound is carried away by saliva, etc. so that the compound cannot become a supply source for the phosphate ion and the calcium ion over a long period of time. Thus, an effect of remineralization cannot be surely obtained.

### SUMMARY OF THE INVENTION

The present invention is aimed to provide a composition for remineralization of a tooth which is able to overcome the problems of the conventional art as described above, and which can be surely diffused into an enamel or dentinal tubule to form a compound within a tooth.

We, the present inventor made extensive and intensive investigations in order to achieve the above-described aim. As a result, it has been found that since a reaction for forming a calcium phosphate compound, a fluoroapatite compound, etc. is extremely fast, in the case of the conventional art composition for remineralization, the enamel or dentinal tubule is filled with a first solution because the first solution to be first applied contains water, and therefore, even when a second solution is applied, the reaction occurs only on a surface layer of the tooth, whereby the compound cannot be formed within the dentinal tubule. Also, it has been mineralized by the inventors that when a solvent of the first solution for remineralization of the tooth, which is to be first applied, is a volatile solvent, as compared with the case where the solvent is water, not only the first solution containing a calcium salt is liable to penetrate into the tooth, but also since the applied solvent is quickly volatilized, without clogging the tubule, the second solution to be subsequently applied is surely diffused into the tubule, to form the compound within the tooth, leading to accomplishment of the present invention.

In addition, it has been found that a solution B containing a fluoride is further effective for the remineralization and that when at least one of solutions A and B contains a surfactant, the solution containing the surfactant is liable to penetrate into the tubule, whereby the compound can be formed in the depth within the tooth. Moreover, it has been mineralized by the inventors that the volatile solvent is preferably one member or a mixture of two or more members selected from ethanol, 2-butanol, and acetone; that the calcium salt is preferably one member or a mixture of two or more members selected from calcium chloride, calcium nitrate, and calcium bromide; that the phosphate is preferably one member or a mixture of two or more members selected from sodium phosphate, potassium phosphate, and lithium phosphate; and that the fluoride is preferably one member or a mixture of two or more members selected from potassium fluoride, sodium fluoride, and lithium fluoride.

### DESCRIPTION OF THE PREFERRED EMBODIMENTS

Specifically, the invention is concerned with a composition for remineralization of a tooth comprising two solutions wherein a solution A containing 1 to 30% by weight of a calcium salt, with the remainder being avolatile solvent; and a solution B is an aqueous solution containing 1 to 30% by weight of a phosphate, the solution A and the solution B being to be applied to a tooth surface in this order, to form a compound within a tooth.

In addition, when the solution B further contains a fluoride in an amount of 0.0001 to 5% by weight, an effect of remineralization by a fluoride ion can be added. Moreover, when at least one of the solution A and the solution B further contains a surfactant in an amount of 0.0005 to 1% by weight, the solution containing the surfactant is more likely penetrated into the tooth, whereby the compound can be formed effectively within the tooth.

The volatile solvent that is used in the solution A of the composition for remineralization of a tooth according to the present invention is a solvent that can quickly penetrate and volatilize during application to a tooth, and it is preferred that the solvent has a boiling point of 100° C. or lower because the solvent can volatilize within a short period of time. As such solvents, are enumerated alcohols, ethers, aldehydes, ketones, and cycloalkanes. More specifically, methanol, ethanol, 1-propanol, 2-propanol, 2-butanol, 2-methyl-2-propanol, diethyl ether, propionaldehyde, acetone, ethyl methyl ketone, cyclopentane, cyclohexane, and so on can be enumerated. It is necessary to use a solvent that is less toxic directly to human bodies. These solvents can be used singly or may be used in admixture of two or more thereof. Of these, three kinds of ethanol, 2-butanol and acetone are preferred, with ethanol alone or a mixed solvent of ethanol and acetone being particularly preferred.

As the calcium salt that is contained in an amount of 1 to 30% by weight in the solution A according to the present invention, any calcium salts that have hitherto been used can be used. Suitable examples include calcium chloride, calcium nitrate, calcium bromide, calcium malate, calcium tartarate, calcium malonate, and calcium succinate, with calcium chloride, calcium nitrate and calcium bromide being preferred. These calcium salts can be used singly or may be used in admixture of two or more thereof.

As the phosphate that is contained in an amount of 1 to 30% by weight in the solution B of the composition for remineralization of a tooth according to the present invention, any phosphates that have hitherto been used can be used. Specific examples include sodium dihydrogenphosphate, disodium hydrogenphosphate, trisodium phosphate, potassium dihydrogenphosphate, dipotassiumhydrogenphosphate, tripotassiumphosphate, lithium dihydrogenphosphate, trilithium phosphate, barium dihydrogenphosphate, barium hydrogenphosphate, magnesium dihydrogenphosphate, magnesium hydrogenphosphate, ammonium dihydrogenphosphate, diammonium hydrogenphosphate, disodium dihydrogenpyrophosphate, tetrasodium pyrophosphate, and tetrapotassium pyrophosphate, with the sodium phosphates, potassiumphosphates and lithiumphosphates being preferred. These phosphates can be used singly or may be used in admixture of two or more thereof.

The reasons why the concentration of the calcium salt in the solution A and the concentration of the phosphate in the solution B, each of which constitutes the composition for remineralization of a tooth according to the present invention, must be 1 to 30% by weight, respectively are as follows. That is, when either one of them is less than 1% by weight, the amount of the compound formed upon application of the both solutions is too little. On the other hand, when either one of them exceeds 30% by weight, the excess of the compound is formed on the surface layer so that the necessary phosphate compound does not possibly reach sufficiently the inside of the tooth. Preferably, the concentrations of the calcium salt in the solution A and of the phosphate in the solution B are 1 to 20% by weight, respectively.

In the composition for remineralization of a tooth according to the present invention, when the solution B further contains a fluoride in an amount of 0.0001 to 5% by weight, the effect for remineralization can further be increased. Suitable examples of the fluoride include aluminum fluoride, antimony fluoride, ammonium fluoride, potassium fluoride, germanium fluoride, sodium fluoride, nickel fluoride, manganese fluoride, and lithium fluoride, with potassium fluoride, sodium fluoride and lithium fluoride being preferred. These fluorides can be used singly or may be used in admixture of two or more thereof.

When the amount of the fluoride in the solution B is less than 0.0001% by weight, the desired effect cannot be obtained. On the other hand, when it exceeds 5% by weight, a danger is high when one erroneously drinks it. Preferably, the amount of the fluoride in the solution B is 0.001 to 1.5% by weight.

In the present invention, when at least one of the solution A and the solution B further contains a surfactant in an amount of 0.0005 to 1% by weight, the solution is liable to more likely penetrate into the dentinal tubule, whereby the compound can be formed in the depth of the tooth.

Examples of the surfactant that can be used in the invention include glycerin fatty acid esters, sucrose fatty acid esters, sorbitan fatty acid esters, polyoxyethylene sorbitan fatty acid esters, propylene glycol fatty acid esters, polyethylene glycol fatty acid esters, soybean phospholipid, polyoxyethylene hardened castor oil, polyoxyethylene polyoxypropylene alkyl ethers, a polyoxyethylene polyoxypropylene block copolymer, alkyl sulfates, and lauromacrogol. When the amount of the surfactant in the solution is less than 0.0005% by weight, the desired effect cannot be obtained. On the other hand, when it exceeds 1% by weight, the formation of the compound is obstructed.

In using the composition for remineralization of a tooth according to the present invention, the prescribed effects can be obtained by the following simple method:

(1) To clean up a tooth surface demineralized by dental caries using a cotton ball or the like and dry the tooth surface by means of air blowing;

(2) To take one or two drops of the solution A on a plate, dip a cotton ball into the drop(s), and then apply the solution A to an affected part;

(3) To volatilize the solvent of the solution A by means of air blowing;

(4) To take one or two drops of the solution B on a plate different from that used for the solution A, dip a cotton ball into the drop(s), and then apply the solution B to the tooth surface having the solution A applied thereto; and

(5) To remove the solution attached to parts other than the desired part using a cotton ball and lightly rinse a mouth.

The composition for remineralization of a tooth according to the present invention will be described below in detail with reference to the following Examples and Comparative Examples, but it should not be construed that the invention is limited to these Examples.

Preparation of a Sample Whose Surface Layer has Been Demineralized:

(1) After extirpation dental pulp, a bovine tooth was embedded with an acrylic resin.

(2) As a test tooth surface, a circle having a diameter of 5 mm was set up on enamel in the lip side, and other exposed parts were masked with a nail varnish.

(3) A sample as prepared in (2) above was immersed in 10 me of a 0.1 M lactic acid buffer solution (pH: 5.0; containing 1.5 mM Ca and 0.9 mM P) at 37° C. for one week. During this period, the lactic acid buffer solution was not changed.

Remineralization Test:

(1) The solution A and the solution B used in the Examples and Comparative Examples were prepared in accordance with the showings of Table 1.

(2) The test tooth surface was cleaned up using a cotton ball and dried by means of air blowing.

(3) The solution A was attached to a cotton ball and applied to the sample surface whose surface layer had been demineralized.

(4) The solvent was volatilized by means of air blowing.

(5) The solution B was attached to a cotton ball and applied to the part having the solution A applied thereto.

(6) Three samples, whose surface layers had been each demineralized but not subjected to a remineralization treatment, were prepared, too.

(7) Each of the samples as prepared in (5) and (6) above was immersed in 10 me of artificial saliva (pH: 7.0) at 37° C. for one month. During this period, the artificial saliva was changed every one week. As the artificial saliva, was used artificial saliva according to the prescription of Pearce, et al. (J. Dent. Res., 66:1518-1522, 1987; 20 mmol/L KCl, 0.5 mmol/L CaCl₂, 1 mmol/L Na₂HPO₄, 5 mmol/L NaHCO₃)

Evaluation Method for Remineralization:

(1) With respect to each sample after the remineralization test, a thin parallel slice having a thickness of about 100 μm was prepared.

(2) With respect to the slice as prepared in (1) above, a mineral concentration distribution was calculated by means of microradiography.

(3) A mineral loss (vol %·μm) was calculated from (2) above.

(4) A remineralization amount of each sample is calculated from the following equation, with a mean value of the mineral losses of the three samples that were not subjected to the treatment with the composition for remineralization being a control. The results are summarized and shown in Table 1.

Remineralization amount (vol %·μm)=(Mineral loss of control)−(Mineral loss of each sample)

TABLE 1

| | | | | | | | | | Com. | Com. | Com. | Com. |
|---|---|---|---|---|---|---|---|---|---|---|---|---|
| | Ex. 1 | Ex. 2 | Ex. 3 | Ex. 4 | Ex. 5 | Ex. 6 | Ex. 7 | Ex. 8 | Ex. 1 | Ex. 2 | Ex. 3 | Ex. 4 |
| Solution A | | | | | | | | | | | | |
| Calcium chloride | 10 | 8 | 10 | 17 | | 13 | | 7 | 1 | | 10 | 10 |
| Calcium nitrate | | 2 | | 2 | 5 | | 2 | | | 5 | | |
| Calcium bromide | | | 1 | 1 | | 2 | 0.5 | | | | | |
| Ethanol | 90 | 45 | 69 | 60 | 40 | 54.7 | 50 | 80 | | | | |
| 2-Butanol | | | | | 10 | | 47.5 | | | | | |
| Acetone | | 45 | 20 | 20 | 45 | 30 | | 13 | | | | |
| Tween 60 | | | | | | 0.3 | | | | 0.3 | | 0.2 |
| Distilled water | | | | | | | | | 99 | 94.7 | 90 | 89.8 |
| Aqueous solution B | | | | | | | | | | | | |
| Potassium | 1 | | | | 2 | | | 0.5 | 1 | | | |
| dihydrogen- | | | | | | | | | | | | |
| phosphate | | | | | | | | | | | | |
| Sodium | | 0.8 | 1.5 | | | 1 | 1 | | | 5 | 1 | 1 |
| dihydrogen- | | | | | | | | | | | | |
| phosphate | | | | | | | | | | | | |
| Lithium | | | | 2 | | | 5 | | | | 5 | |
| dihydrogen- | | | | | | | | | | | | |
| phosphate | | | | | | | | | | | | |
| Dipotassium | 10 | | | 15 | 5 | | | | | | | |
| hydrogen- | | | | | | | | | | | | |
| phosphate | | | | | | | | | | | | |
| Disodium | | 5 | 10 | | | 7 | | 3 | | | 5 | 5 |
| hydrogen- | | | | | | | | | | | | |
| phosphate | | | | | | | | | | | | |
| Tripotassium | | | | | 1 | | | | | | | |
| phosphate | | | | | | | | | | | | |
| Sodium fluoride | | 0.2 | | | | 0.8 | 0.004 | 0.3 | | | 1 | 0.5 |
| Zinc fluoride | | | | | | | | | 0.1 | | | |
| PEP-101 | | | | | | 0.2 | 0.2 | | | 0.1 | | 0.1 |
| Distilled water | 89 | 94 | 88.5 | 83 | 92 | 91 | 93.796 | 96.2 | 98.9 | 94.9 | 88 | 93.4 |
| Remineralization | 5300 | 5900 | 3100 | 4600 | 2600 | 6200 | 3500 | 5100 | 500 | 300 | 900 | 1200 |
| amount (vol % · μm) | | | | | | | | | | | | |

PEP-101: Polyoxyethylene (105) polyoxypropylene (5) glycol (made by Freund Inc.)
Tween 60: Polyoxyethylene (20) sorbitan monostearate (made by ICI)

As is clear from Table 1, it is evident that Examples 1 to 8 using the composition for remineralization of a tooth according to the present invention are larger in the remineralization amount and are increased in the effect for remineralization, as compared with Comparative Examples 1 to 4 using the conventional art for remineralization.

As has been described above in detail, the composition for remineralization according to the present invention comprises two solutions wherein a solution A containing 1 to 30% by weight of a calcium salt, with the remainder being a volatile solvent and a solution B is an aqueous solution containing 1 to 30% by weight of a phosphate, the solution A and the solution B being to be applied to a tooth surface in this order, to form a compound within a tooth. The formed compound remains within the tooth and gradually dissolves to supply a phosphate ion and a calcium ion from the inside of the tooth. Thus, the present invention can surely accelerate the remineralization of the tooth as compared with the conventional art for remineralization.

When the solution B further contains a fluoride in an amount of 0.0001 to 5% by weight, since the compound that remains within the tooth and gradually dissolves also supplies a fluoride ion, the effect of remineralization by the fluoride ion can be added. Moreover, when at least one of the solution A and the solution B further contains a surfactant in an amount of 0.0005 to 1% by weight, the solution containing the surfactant is more likely penetrated into the tooth, whereby the compound can be formed effectively within the tooth.

In the light of the above, the present invention having various effects as described above is greatly valuable in contributing to the dental field.

While the invention has been described in detail and with reference to specific embodiments thereof, it will be apparent to one skilled in the art that various changes and modifications can be made therein without departing from the spirit and scope thereof.

What is claimed is:

1. A composition for remineralization of a tooth comprising two solutions wherein a solution A contains 1 to 30% by weight of a calcium salt, with the remainder being a volatile solvent and a solution B is an aqueous solution containing 1 to 30% by weight of a phosphate, the solution A and the solution B being to be applied to a tooth surface in this order, to form a compound within a tooth.

2. The composition for remineralization of a tooth as claimed in claim 1, wherein the solution B further contains a fluoride in an amount of 0.0001 to 5% by weight.

3. The composition for remineralization of a tooth as claimed in claim 1 or 2, wherein at least one of the solution A and the solution B further contains a surfactant in an amount of 0.0005 to 1% by weight.

4. The composition for remineralization of a tooth as claimed in any one of claims 1 to 3, wherein the volatile solvent is one member or a mixture of two or more members selected from ethanol, 2-butanol and acetone.

5. The composition for remineralization of a tooth as claimed in any one of claims 1 to 4, wherein the calcium salt is one member or a mixture of two or more members selected from calcium chloride, calcium nitrate and calcium bromide.

6. The composition for remineralization of a tooth as claimed in any one of claims 1 to 5, wherein the phosphate is one member or a mixture of two or more members selected from sodium phosphate, potassium phosphate and lithium phosphate.

7. The composition for remineralization of a tooth as claimed in any one of claims 2 to 6, wherein the fluoride is one member or a mixture of two or more members selected from potassium fluoride, sodium fluoride and lithium fluoride.
